(19) United States
(12) Patent Application Publication
(10) Pub. No.: US 2017/0065189 A1
(43) Pub. Date: Mar. 9, 2017

(54) SYSTEMS AND METHODS FOR PERFORMING AN OBJECTIVE ANALYSIS OF AIR PLETHYSMOGRAPHY WAVEFORM

(71) Applicant: ProMedica Health System, Inc., Toledo, OH (US)

(72) Inventors: Robert SCISSONS, Toledo, OH (US); Anthony COMEROTA, Toledo, OH (US); Fedor LURIE, Toledo, OH (US); Robin ACINO, Toledo, OH (US)

(21) Appl. No.: 15/122,444

(22) PCT Filed: Mar. 3, 2015
(86) PCT No.: PCT/US2015/018411

§ 371 Date: Aug. 30, 2016

Related U.S. Application Data
(60) Provisional application No. 61/947,102, filed on Mar. 3, 2014.

(51) Int. Cl. A61B 5/0295; A61B 5/02; A61B 5/00

(52) U.S. Cl. CPC A61B 5/0295; A61B 5/7282; A61B 5/742; A61B 5/02007

(57) ABSTRACT

One aspect of the present disclosure relates to a system that can provide an objective interpretation of an air plethysmographic waveform (APW). An APW taken from a subject can be received (e.g., from an device recording the APW during an air plethysmograph test). Based on an analysis of the APW, a characteristic of the APW can be determined. A suggested diagnosis for the subject can be provided based on the characteristic of the APW.

### RELATED APPLICATIONS

This application claims the benefit of U.S. Provisional Application No. 61/947,102, filed Mar. 3, 2014, entitled SYSTEM AND METHOD FOR RECORDING AND ANALYSIS OF AIR PLYTHSMOGRAPHIC PULSE VOLUME RECORDING WAVEFORMS (PVRs), which is incorporated herein by reference in its entirety.

### FIELD OF THE DISCLOSURE

The present disclosure relates generally to air plethysmography and, more specifically, to systems and methods that can perform an objective analysis of an air plethysmography waveform (APW).

### BACKGROUND OF THE DISCLOSURE

Air plethysmography is a non-invasive medical test that can measure changes in volume in different parts of a subject's body. Air plethysmography has long been an intricate component of the noninvasive physiologic evaluation of peripheral artery disease (PAD). While air plethysmography waveform (APW) criteria can indicate varying levels of PAD severity, these criteria are largely subjective and based on pattern recognition.

### SUMMARY OF THE DISCLOSURE

The present disclosure relates generally to air plethysmography and, more specifically, to systems and methods that can perform an objective analysis of an air plethysmography waveform (APW).

In an aspect of the present disclosure, a system is provided. For example, the system can provide an objective analysis of an APW. The system can include a memory to store computer-executable instructions and a processor to execute the computer-executable instructions. An APW waveform taken from a subject can be received. Based on an analysis of the APW, a characteristic of the APW can be determined. A suggested diagnosis for the subject can be provided based on the characteristic of the APW.

According to another aspect of the present disclosure, a method is provided. The method can, for example, provide an objective analysis of an APW. One or more acts of the method can be represented by computer-executable instructions that can be executed by a system that includes a processor. An APW taken from a subject can be received. Based on an analysis of the APW, a characteristic of the APW can be determined. Based on the characteristic of the APW, a suggested diagnosis for the subject can be provided.

In another aspect of the present disclosure, a device is provided. For example, the device can provide an objective analysis of an APW. The device can include an air plethysmograph recording device coupled to a computing device. The computing device can include a memory to store computer-executable instructions and a processor to execute the computer-executable instructions. An APW taken from a subject can be received from the air plethysmograph recording device. A characteristic of the APW can be determined based on an analysis of the APW. A suggested diagnosis for the subject can be provided based on the characteristic of the APW.

### BRIEF DESCRIPTION OF THE DRAWINGS

The foregoing and other features of the present disclosure will become apparent to those skilled in the art to which the present disclosure relates upon reading the following description with reference to the accompanying drawings, in which:

FIG. 1 is a block diagram of a system that can record and perform an objective analysis of an air plethysmography waveform (APW) in accordance with an aspect of the present disclosure;

FIG. 2 is a block diagram of an example of the analysis device of the system shown in FIG. 1;

FIG. 3 is a block diagram of an example of the APW analyzer of the analysis device shown in FIG. 2;

FIGS. 4-5 are process flow diagrams showing methods for performing an objective analysis of an APW in accordance with other aspects of the present disclosure;

FIGS. 6-8 illustrate examples of features that best identify inflow disease (>50% diameter);

FIGS. 9-13 illustrate characteristics of an APW in extremities with significant 50%) obstruction in the common femoral artery or above (INFLOW) and/or in the femoropopliteal arteries (OUTFLOW); and

FIGS. 14-20 illustrate a comparison between subjective and objective APW analysis methods.

### DETAILED DESCRIPTION

### I. Definitions

In the context of the present disclosure, the singular forms “a,” “an” and “the” can also include the plural forms, unless the context clearly indicates otherwise.

The terms “comprises” and/or “comprising,” as used herein, can specify the presence of stated features, steps, operations, elements, and/or components, but do not preclude the presence or addition of one or more other features, steps, operations, elements, components, and/or groups.

As used herein, the term “and/or” can include any and all combinations of one or more of the associated listed items.

Additionally, although the terms “first,” “second,” etc. may be used herein to describe various elements, these elements should not be limited by these terms. These terms are only used to distinguish one element from another. Thus, a “first” element discussed below could also be termed a “second” element without departing from the teachings of the present disclosure. The sequence of operations (or acts/steps) is not limited to the order presented in the claims or figures unless specifically indicated otherwise.

As used herein, the term “air plethysmography” generally refers to a non-invasive medical test that can measure changes in volume in a portion of a subject's body (e.g., based on an air plethysmography waveform (APW)). In some instances, air plethysmography can be used to measure the changes in volume in a patient's leg, calf, thigh, or the like. For example, air plethysmography can be used in the noninvasive physiologic evaluation of peripheral artery disease (PAD).

As used herein, the terms “APW” and “PVR waveform” can refer to one or more waveforms that can be used to detect changes in volume in the portion of the subject's body. For example, the APW can be used as a measure of perfusion within the subject's body. In some instances, the APW can include one or more single-level waveforms. In other instances, the APW can include one or more multilevel waveforms.

As used herein, the terms “characteristic,” “feature,” and “parameter” of the APW can refer to a numerical attribute of the APW. For example, characteristics of the APW can include a presence of a dicrotic notch, a relative amplitude, an acceleration time, a deceleration time, and a downslope curvature.

As used herein, the term “objective analysis” can refer to an empirical study based on observable phenomenon. In contrast to a subjective analysis, an objective analysis does not rely solely on an interpretation of a medical professional. In some instances, the objective analysis can utilize a standardized measure. In other instances, the objective analysis can be performed by a computerized device (e.g., a processor).

As used herein, the terms “subject” and “patient” can refer to any warm-blooded organism including, but not limited to, a human being, a pig, a rat, a mouse, a dog, a cat, a goat, a sheep, a horse, a monkey, an ape, a rabbit, a cow, etc.

As used herein, the term “operative communication” can include, but is not limited to, a communicative relationship between devices, logic, or circuits, including wired and wireless relationships. Direct and indirect electrical, electromagnetic, and optical connections are examples of connections that facilitate operative communications. Two devices are in operative communication if an action from one causes an effect in the other, regardless of whether the action is modified by some other device. For example, two devices in operable communication may be separated by one or more of the following: i) amplifiers, ii) filters, iii) transformers, iv) optical isolators, v) digital or analog buffers, vi) analog integrators, vii) other electronic circuitry, viii) fiber optic transceivers, ix) Bluetooth communications links, x) IEEE 802.11 communications links, xi) satellite communication links, xii) gateways, repeaters, routers, and hubs, xiii) wired or wireless networks, xiv) mobile communications towers, and xv) other wired or wireless communication links. Operative communication may be facilitated by and exist between devices using, for example, the internet or service provider networks. As another example, an electromagnetic sensor is in operative communication with a signal if it receives electromagnetic radiation from the signal. As a final example, two devices not directly connected to each other, but both capable of interfacing with a third device, e.g., a central processing unit (CPU), are in operative communication.

### II. Overview

The present disclosure relates generally to air plethysmography. For example, air plethysmography has long been a component of the non-invasive physiologic evaluation of peripheral artery disease (PAD). Previously, in the physiologic evaluation, the air plethysmography waveform (APW) (produced during air plethysmography) has been analyzed according to largely subjective guidelines (e.g., based on pattern recognition) to determine PAD severity. The systems and methods of the present disclosure perform an objective analysis of the APW. In contrast to the historical subjective analysis using pattern recognition, the objective analysis of the systems and methods described herein does not rely solely on an interpretation of a medical professional.

The objective analysis can be performed on the APW taken from a patient. Based on the analysis of the APW, a characteristic of the APW can be determined. A suggested diagnosis for the subject can be provided (e.g., for review by a medical professional) based on the characteristic of the APW.

### III. Systems

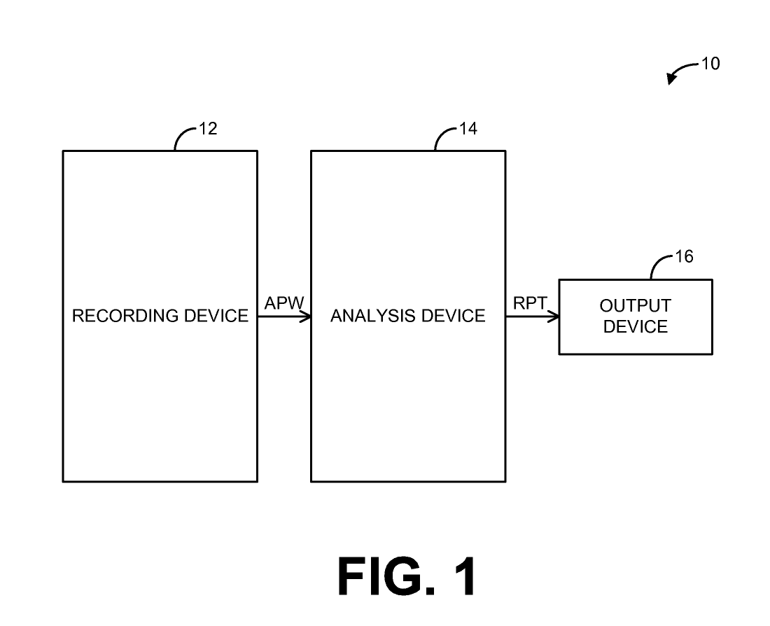

One aspect of the present disclosure, as illustrated in FIG. 1, can include a system 10 that can record and perform an objective analysis of an air plethysmography waveform (APW). For example, the system 10 can facilitate the diagnosis of a vascular disorder (e.g., peripheral artery disease (PAD)) in a patient. In this example, the probability of a proximal obstruction or a distal obstruction can be determined. Additionally or alternatively, a PAD severity and/or disease level independent of systolic pressure measurement and pressure indices data can be determined and/or a success and/or potential outcome of patients with a PAD intervention can be determined, However, the system 10 can be used in applications beyond diagnosis and monitoring of PAD.

The system can include a recording device 12 that can record at least one APW; an analysis device 14 that can receive the APW, perform the objective analysis of the at least one APW, and produce a report (RPT) of the objective analysis; and an output device 16 to output at least a portion of the report (RPT). The recording device 12 can be operative coupled to the analysis device 14. The analysis device 14 can be operatively coupled to the output device 16.

As an example, the recording device 12 can include an air-filled cuff that can be attached to an extremity (e.g., a patient's arms, legs, etc.). The cuff can be communicatively coupled to the analysis device 14, in some examples. In other examples, the cuff can be communicatively coupled to a recording computer, which can be coupled to the analysis device 14. The recording device 12 can record APWs that can indicate circulatory capacity from one or more regions of the body. For example, the recording device 12 can record the APW from a single region of the body (e.g., a single extremity). In another example, the recording device 12 can record multiple APWs from multiple points within the region (e.g., segmental recordings can be taken from the thigh, the calf, and the ankle of the same lower leg). In a further example, the recording device 12 can record multiple APWs from different regions and/or different extremities.

The analysis device 14 can receive the one or more APWs from the recording device 12 and perform the objective analysis of the APWs. The analysis device 14 can include one or more computing devices. For example, the analysis device 14 described herein can be embodied at least in part in hardware and/or in software (including firmware, resident software, micro-code, etc.). Furthermore, aspects of the analysis device 14 can take the form of a computer program product on a computer-usable or computer-readable storage medium having computer-usable or computer-readable program code embodied in the medium for use by or in connection with an instruction execution system.

The analysis device 14 can include various systems and subsystems, including a personal computer, a laptop computer, a workstation, a computer system, an appliance, an application-specific integrated circuit (ASIC), a server, a server blade center, a server farm, etc. For example the analysis device 14 can includes a system bus, a processor or processing unit, memory devices (e.g., a system memory and/or additional memory devices), a communication interface (e.g., a network interface to communicate with devices, such as the recording device 12 and the output device 16), a communication link, and an input device (e.g., a keyboard and/or a mouse). The system bus can be in communication with the processor (e.g., a computing device that executes a set of instructions to implement the operations of examples disclosed herein), the memory devices (including the system memory and the additional memory devices, such as a hard disk drive, server, stand alone database, or other non-volatile memory), and the communication interface.

The memory devices can include a non-transitory computer-readable memory (e.g., any non-transitory medium that is not a transitory signal and can contain or store the program for use by or in connection with the instruction or execution of a system, apparatus, or device) that can store computer program instructions that can implement the functionality of the analysis device 14 (e.g., perform the objective analysis). These computer program instructions can be provided to the processor to produce a machine, such that the instructions, which execute via the processor, create a mechanism for implementing the functions of analysis device 14. For example, the computer program instructions can direct a computer or other programmable data processing apparatus to function in a particular manner, such that the computer program instructions stored in the non-transitory computer-readable memory produce an article of manufacture including instructions, which implement the function specified in the block diagrams and associated description. Upon execution, the computer program instructions can cause a series of operational steps to be performed by the analysis device 14 to produce a computer-implemented process for implementing objective analysis.

The output device 16 can receive the report (RPT) of the objective analysis. In some instances, the output device 16 can display at least a portion of the report (RPT). For example, the output device 16 can include a visual display (e.g., a monitor) that can display the portion of the report visually. As another example, the output device 16 can include an audio display (e.g., a speaker) that can display the portion of the report as an audio message. As another example, the output device 16 can include a printer that can print a visual image of the report (RPT).

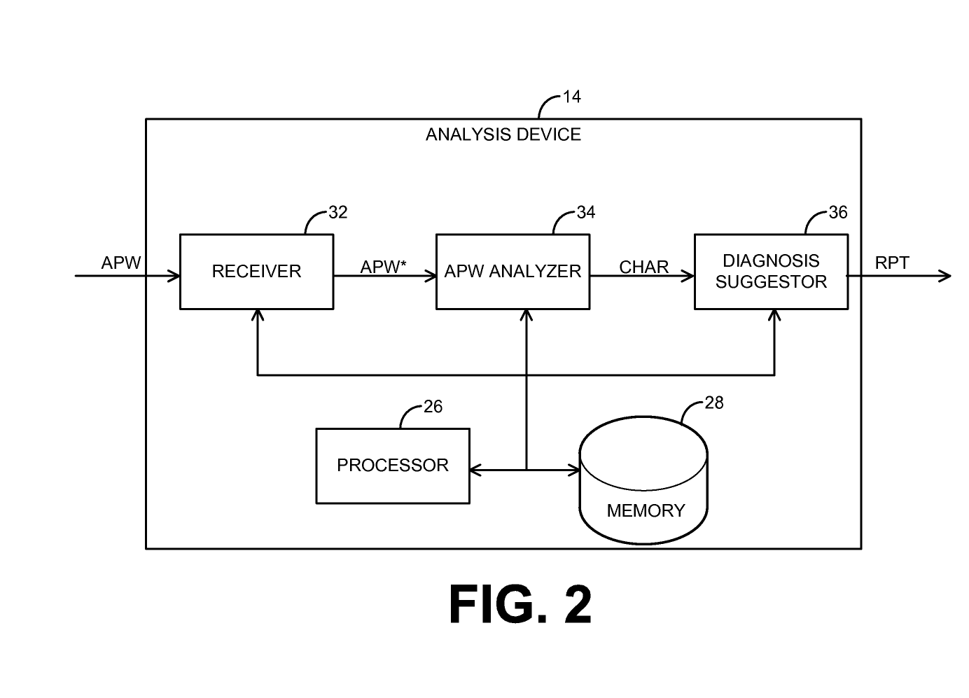

FIG. 2 shows an example configuration of the analysis device 14 of FIG. 1. The analysis device 14 can receive one or more APWs (e.g., from the recording device 12) and output a report (RPT) (e.g., to the output device). As described above, the analysis device 14 can include a memory 28 that stores computer-executable instructions and a processor 26 to execute the computer-executable instructions. Upon execution by the processor 26, the computer-executable instructions can implement components of the analysis device 14, including a receiver 32, an APW analyzer 34, and a diagnosis suggestor 36.

The receiver 32 can receive the one or more APWs (e.g., from the recording device 12). In some instances, the one or more APWs can originate from one area in a patient's body. In other instances, the one or more APWs can come from different parts of the area of the patient's body (e.g., segmental analysis). In still other instances, the one or more APWs can come from different areas of the patient's body. The receiver 32 can perform preprocessing on the APW to produce a new APW* that can be sent to the APW analyzer. For example, the receiver 32 can perform preprocessing to remove extra data from the APW. As another example, the receiver 32 can split the one or more APWs into a waveform that includes a format that is easier for the APW analyzer 34 to analyze (e.g., single APWs). Although the receiver 32 does not necessarily perform the preprocessing, APW* is shown as being fed to the APW analyzer 34 to avoid confusion.

The APW analyzer 34 can receive the APW* and determine a characteristic (CHAR) of the APW*. For example, the APW analyzer 34 can perform an automated (e.g., computer-implemented, without interpreter bias) analysis of the APW*. The characteristic (CHAR) can be any feature of the APW* that leads to a diagnosis. For example, the characteristic (CHAR) can be extracted from the APW*. For example, the characteristic (CHAR) can be: a relative amplitude parameter; an acceleration time parameter; a down-slope curvature parameter; a gain parameter; a deceleration curve parameter; and/or another feature that can be extracted from the APW waveform.

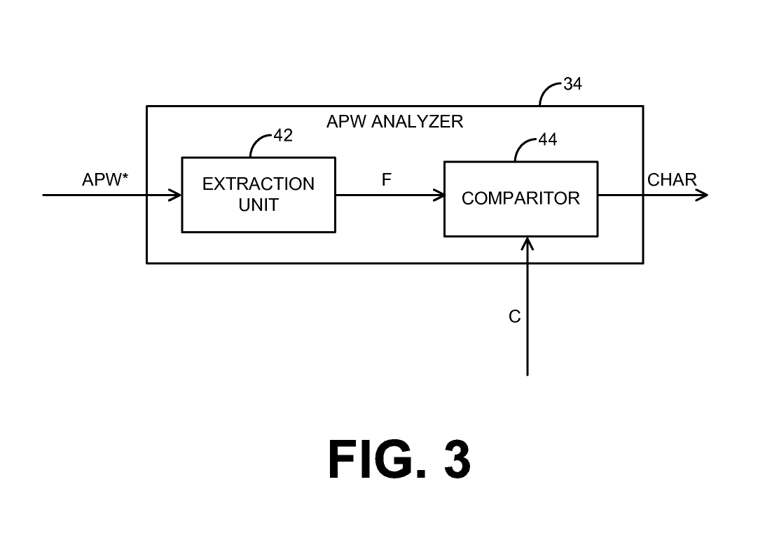

An example of the APW analyzer 34 is shown in FIG. 3. For example, the APW* can be received by an extraction unit 42, which can extract one or more features (F) of the APW*. The extraction unit 42 can, for example, be programmed to extract a certain one or more features from the APW* (e.g., the feature can be predefined by a medical professional or at the factory). The extraction unit 42 can feed the feature (F) to the comparator 44. In some instances, the comparator 44 can receive a control (C) to compare the feature (F) against. In some instances, the comparison can be against data recorded from another area of the subject's body. In other instances, the comparison can be against historical data recorded at a previous time from other patient(s) (e.g., an average of previous patients). In further instances, the comparison can be against numerical controls that are not necessarily from another APW. However, in each instance, the comparison can occur without requiring a standardization of the APW (e.g., normalization with respect to a baseline value). Instead, the comparator 44 compares the feature (F) (e.g., the presence of absence of a characteristic of the APW) to the control (C), which can determine the presence or absence of the characteristic. As an example, the feature (F) can be a presence or absence of a down-slope curvature. The control (C) can either have or not have a down-slope curvature. The comparator 44 can determine the characteristic (CHAR) based on the comparison. For example, the characteristic (CHAR) can include the feature (F), the control (C), and/or the result of the comparison by the comparator 44.

Referring again to FIG. 2, a diagnosis suggestor 36 can suggest a diagnosis based on the characteristic (CHAR) of the APW*. For example, the diagnosis can be suggested in the report (RPT) (e.g., that is sent to the output device). As another example, the diagnosis can include a probability that the subject has a disease or medical condition based on the characteristic (CHAR). For example, the report (RPT) can indicate a location of an obstruction to blood flow in a subject's vasculature. The report (RPT) can indicate a certain probability of the obstruction being in one or more areas within the vasculature, which can aid in the diagnosis of the location of the obstruction.

### IV. Methods

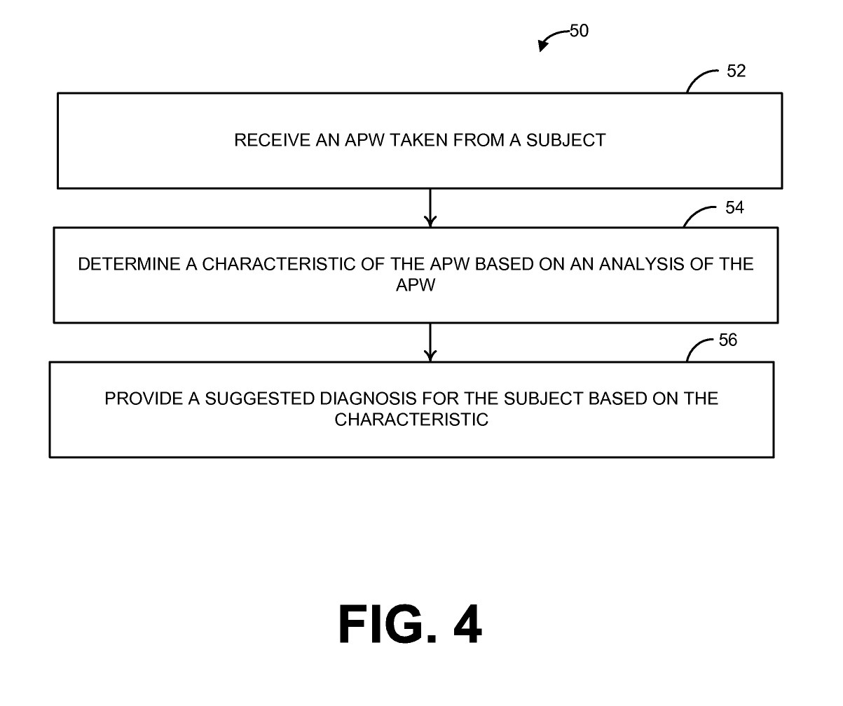

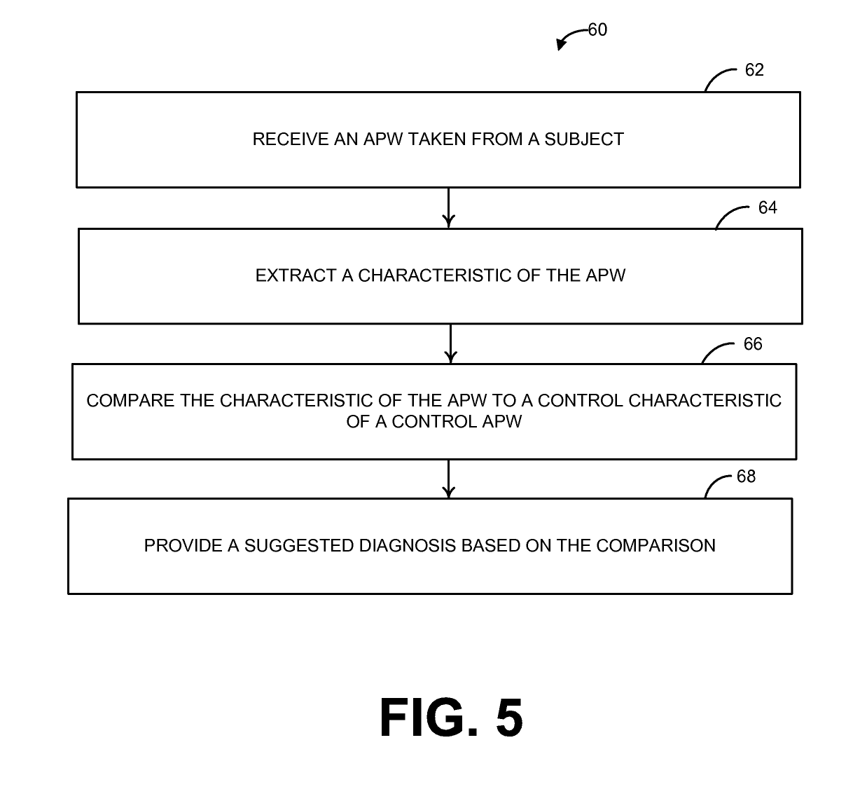

Another aspect of the present disclosure can include methods for performing an objective analysis of an APW. One example of such a method 50 is shown in FIG. 4. Another example of such a method 60 is shown in FIG. 5. The methods 50 and 60 are illustrated as process flow diagrams with flowchart illustrations. For purposes of simplicity, the methods 50 and 60 are shown and described as being executed serially; however, it is to be understood and appreciated that the present disclosure is not limited by the illustrated order as some steps could occur in different orders and/or concurrently with other steps shown and described herein. Moreover, not all illustrated aspects may be required to implement the methods 50 and 60.

One or more blocks of the respective flowchart illustrations, and combinations of blocks in the block flowchart illustration, can be implemented by computer program instructions. These computer program instructions can be stored in memory and provided to a processor of a general purpose computer, special purpose computer, and/or other programmable data processing apparatus to produce a machine, such that the instructions, which execute via the processor of the computer and/or other programmable data processing apparatus, create mechanisms for implementing the steps/acts specified in the flowchart blocks and/or the associated description. In other words, the steps/acts can be implemented by a system comprising a processor that can access the computer-executable instructions that are stored in a non-transitory memory.

The methods 50 and 60 of the present disclosure may be embodied in hardware and/or in software (including firmware, resident software, micro-code, etc.). Furthermore, aspects of the present disclosure may take the form of a computer program product on a computer-usable or computer-readable storage medium having computer-usable or computer-readable program code embodied in the medium for use by or in connection with an instruction execution system. A computer-usable or computer-readable medium may be any non-transitory medium that can contain or store the program for use by or in connection with the instruction or execution of a system, apparatus, or device.

Referring now to FIG. 4, an aspect of the present disclosure can include a method 50 for performing an objective analysis of an APW. At element 52, the APW taken from a subject can be received (e.g., by receiver 32 from recording device 12). At element 54, a characteristic (e.g., CHAR) of the APW can be determined based on an analysis of the APW (e.g., by APW analyzer 34). At element 56, a suggested diagnosis can be provided (e.g., by diagnosis suggestor 36) based on the characteristic (CHAR). For example, the suggested diagnosis can be provided in a report (e.g., RPT) The report can include data related to the analysis of the APW and/or the suggested diagnosis.

Referring now to FIG. 5, another aspect of the present disclosure can include another method 60 for performing the objective analysis of the APW. Similarly to element 52, at element 62, an APW taken from a subject can be received (e.g., by receiver 32 from recording device 12). At 64, a characteristic (e.g., feature (F)) can be extracted from the APW (e.g., by extraction unit 42 of APW analyzer 34). At 66, the characteristic of the APW (e.g., feature (F)) can be compared (e.g., by comparator 44 of APW analyzer 34) to a control characteristic (e.g., control (C)) of a control APW. However, the control characteristic need not come from an actual APW and, instead, may be a numerical control. At 68, similar to element 56, a suggested diagnosis can be provided (e.g., by diagnosis suggestor 36) based on the comparison.

### IV. Experimental

The following examples are for the purpose of illustration only and is not intended to limit the scope of the appended claims.

### Example 1

This example illustrates the features that best identify inflow disease (>50% diameter).

### Methods

Non-diabetic vascular laboratory patients undergoing segmental pressures, APW analysis and arteriography within thirty days of the noninvasive procedure were investigated. Patients with prior aortoiliac intervention were excluded from the analysis. Institutional review board approval for a retrospective review was attained.

Systolic limb pressures and air plethysmographic waveforms were obtained with Unetixs Multilab 2-LHS and 2-CP physiologic testing equipment and 8 or 5 MHz bi-directional, CW Doppler transducers (Unetixs Vascular, Inc., North Kingstown, R.I.). Segmental upper arm brachial, and lower extremity thigh, below knee, posterior tibial and dorsalis pedis ankle pressures were performed with appropriately sized pressure cuffs in the supine position following 10-minutes of rest. Thigh pressures were obtained at the upper most segment of the thigh; bladder size was 11.4×40.6 cm. Upper thigh and below knee (calf) pressure measurements were obtained from the ankle level posterior tibial (PT) and dorsalis pedis (DP) artery with the highest systolic pressure. Thigh, calf and ankle pressure indices referenced the higher of the two brachial pressure measurements.

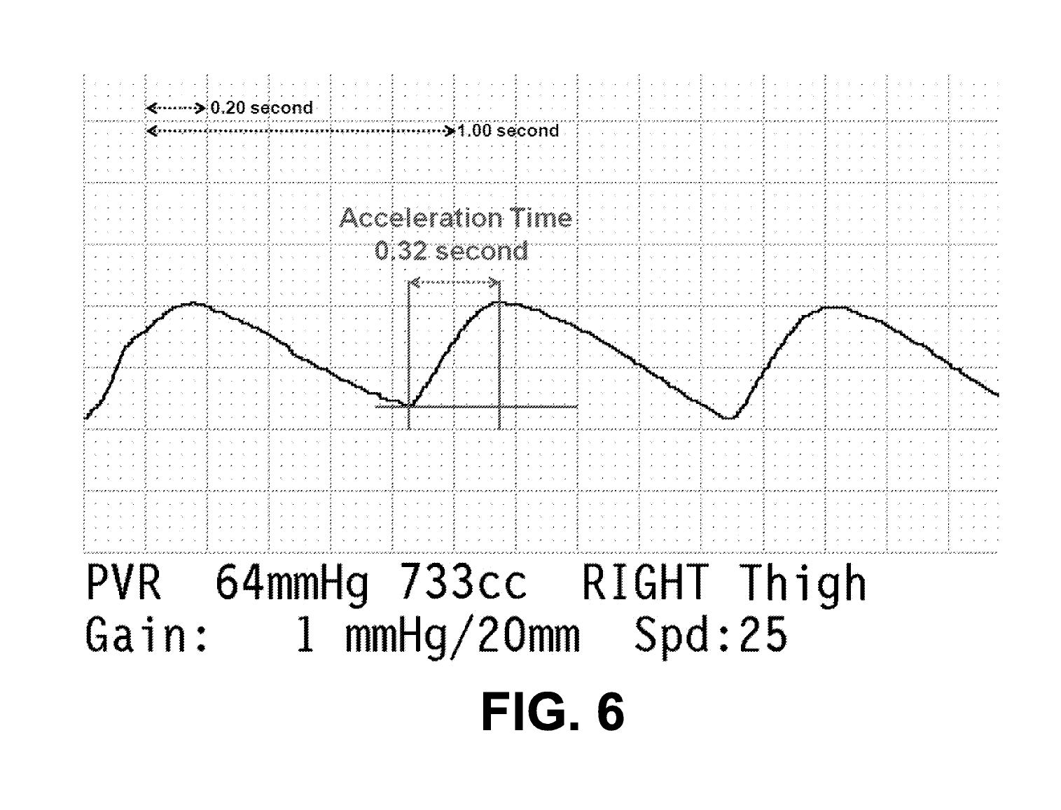

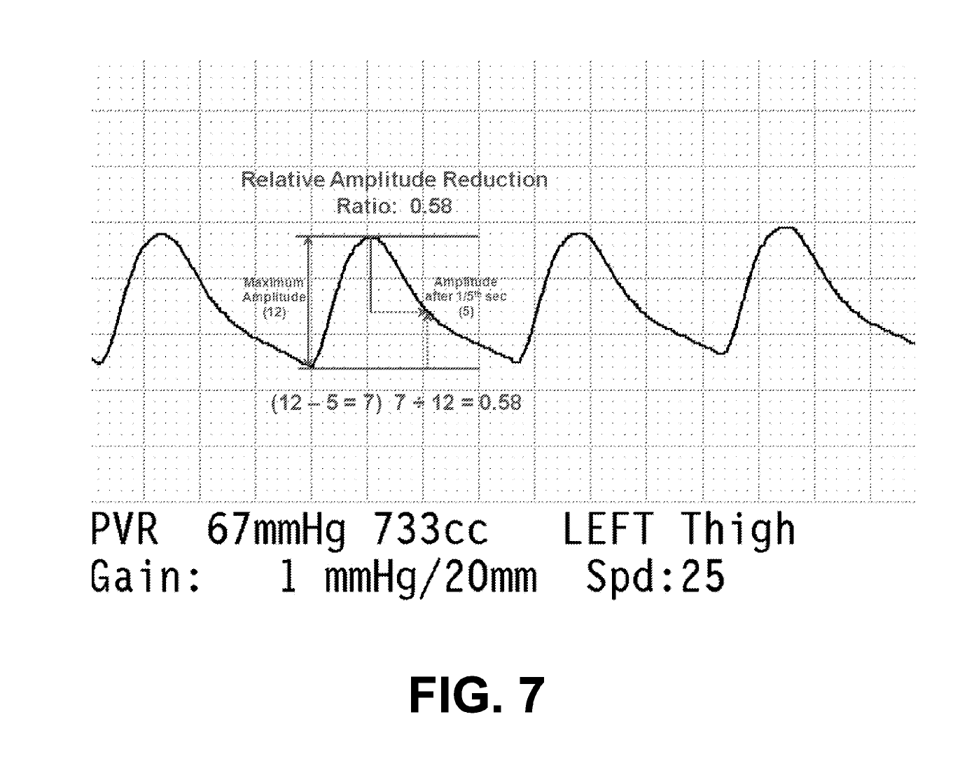

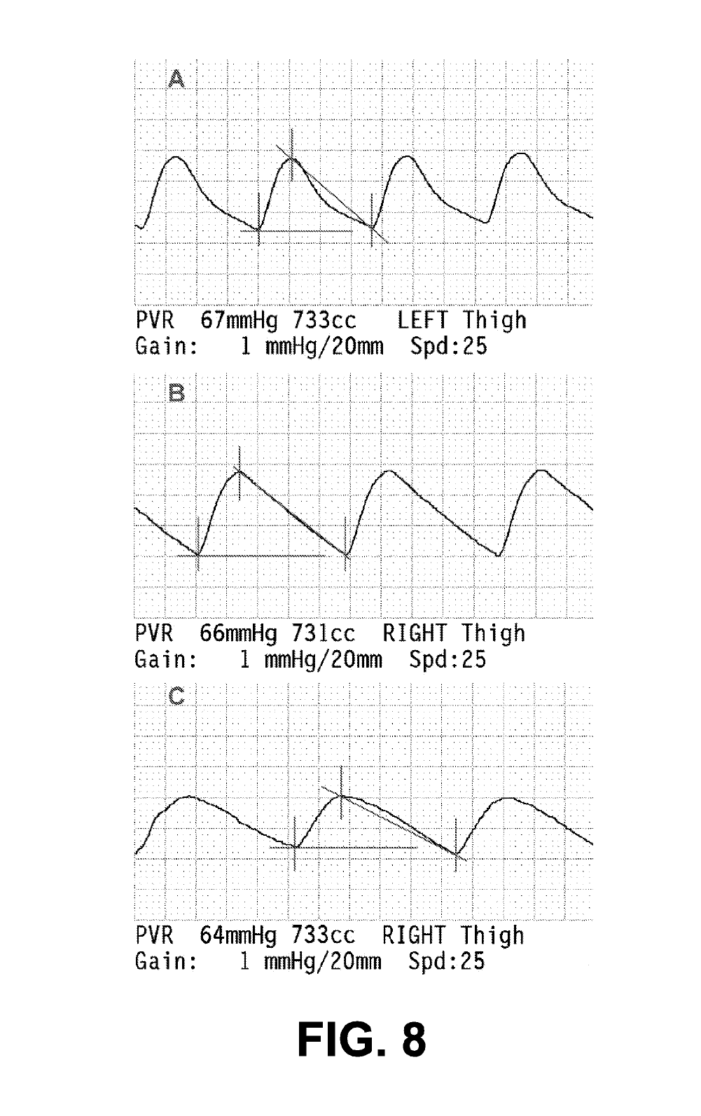

Calibrated pulse volume recording was employed to standardize cuff pressure and air volume. Waveform gain varied but was standardized for each patient at the thigh, calf and ankle levels; chart recorder speed was 25 mm/sec for all measurements. The upper thigh waveform most representative of the displayed waveform cycle was evaluated for the following characteristics:
1. Acceleration time (TIME), in seconds, from the onset of systole to mid-peak systole and rounded off to the nearest whole number (shown in FIG. 6).
2. Relative amplitude reduction (RAR) expressed as a ratio: maximum peak systolic waveform amplitude from the onset of systole minus the downslope waveform amplitude one-fifth of a second after mid-peak systole, divided by maximum peak systolic waveform amplitude and rounded off to the nearest whole number (shown in FIG. 7).
3. Down-slope curvature (CURV) based on the direction of the dicrotic limb curvature in relationship to a reference line drawn from mid-peak systole to end diastole.
   a. Inward CURV—inward deflection of 50% or greater from baseline (shown in FIG. 8A).
   b. Even CURV—inward and/or outward deflection of less than 50% (shown in FIG. 8B).
   c. Outward CURV—outward deflection of 50% or greater from baseline (shown in FIG. 8C).

Two physicians shared responsibility for interpreting the arteriograms and were blinded to the noninvasive data. Each limb was classified as negative or positive for hemodynamically significant INFLOW disease. Positive INFLOW was defined as greater than 50 percent diameter stenosis or occlusion in one or more of the following vessels: abdominal aorta, common iliac, external iliac or common femoral artery.

Statistical analysis for waveform and pressure data was performed using SAS 9.2 (TS level 2M3, XP_PRO platform, SAS Institute, Cary, N.C.). Descriptive statistics are expressed as mean±standard deviation (SD). For all analyses, a P value of less than 0.05 was used to define statistical significance.

### Results

A total of 30 patients were analyzed; 63% males (19/30) and 37% females (11/30). The average age was 71 years old, ranging in age from 43 to 91 years. There were 16 bilateral and 14 unilateral extremity evaluations with appropriate clinical indications: claudication (53%), limb ischemia (13%), rest pain (23%) and ulceration (10%).

Forty-six extremities were investigated; 48% (22/46) right and 52% (24/46) left. Combined average of all extremity posterior tibial (PT) and dorsalis pedis ankle-brachial index (ABI) was 0.64. Average thigh waveform gain was five (range 3 to 12). Thigh waveform characteristics representative of extremities negative, borderline and positive for hemodynamically significant (>50% diameter) INFLOW are summarized in the table below.

| Waveform | Waveform | Relative | INFLOW |
|---|---|---|---|
| Down-Slope | RiseTime (TIME) | Amplitude | ≧50% diameter |
| (CURV) | sec | Reduction (RAR) | reduction |
| In | <0.28 | | Negative |
| In | >0.28 | | Negative |
| Even | <0.28 | | Negative |
| In | 0.28 | ≧0.50 | Borderline |
| | | | Negative |
| Even | 0.28 | >0.50 | Borderline |
| | | | Negative |
| In | 0.28 | <0.50 | Borderline Positive |
| Even | 0.28 | ≦0.50 | Borderline Positive |
| Even | >0.28 | | Positive |
| Out | | | Positive |

Thirty extremities (65%) were negative for INFLOW: one insignificant (<50%) extremity, eight with single level femoropopliteal disease, five with outflow (tibioperoneal) disease and sixteen extremities with concomitant femoropopliteal and tibioperoneal disease. Mean brachial to upper thigh cuff pressure differences amplitude was −13.63±46, standard deviation (SD). Mean thigh waveform amplitude was 16.43±1.2, TIME was 6.13±1.2, and RAR was 0.48±0.13. Percent waveform downslope CURVE was 63% (19/30) inward, 33% (10/30) even and 3% (1/30) outward.

Sixteen extremities (35%). were positive for INFLOW: four single level inflow, three multilevel with associated femoropopliteal disease, four multilevel with associated outflow disease, and five multilevel with concomitant femoropopliteal and outflow disease. Mean brachial to upper thigh cuff pressure differences amplitude was −24.19±32.7, standard deviation (SD). Mean thigh waveform amplitude was 10.38±4.01, TIME was 7.88±1.02, and RAR was 0.39±0.17. Percent waveform downslope CURVE was 25% (4/16) inward, 33% (8/16) even and 25% (4/16) outward.

Normal arteries or insignificant INFLOW was defined by thigh APWs with >0.28<sec TIME and inward CURV or <0.28 sec TIME and even CURV. Borderline INFLOW was defined: 0.28 sec TIME, RAR>0.50<and inward or even CURV. Significant INFLOW was defined with >0.28 sec TIME and outward or even CURV. Sensitivity for defining INFLOW was 94% (15/16) and specificity 81% (26/30); PPV was 79% (15/19) and NPV 96% (26/27).

If borderline extremities were included in the negative INFLOW category, sensitivity would be 63% (10/16), specificity 97% (29/30), PPV would be 91% (10/11) and NPV 83% (29/35). If borderline extremities were included in the positive INFLOW category, sensitivity would be 94% (15/16) and specificity 73% (22/30); PPV is 65% (15/23) and NPV 96% (22/23).

### Example 2

This example illustrates a study of the characteristics of the APW in extremities with significant (≧50%) obstruction in the common femoral artery or above (INFLOW) and/or in the femoropopliteal arteries (OUTFLOW).

### Methods

Vascular laboratory patients who underwent segmental arterial evaluations and who had angiography within 60 days were included in the study. Patients with a prior INFLOW or OUTFLOW interventions were excluded. This study was approved by the hospital system's institutional review board.

The noninvasive arterial evaluation was performed by certified vascular sonographers in an Intersocietal Accreditation Commission (IAC) accredited vascular laboratory under the supervision of. an American Registry for Diagnostic Medical Sonography (ARDMS) Registered Vascular Technologist (RVT). Evaluations were reviewed by an ARDMS Physician in Vascular Interpretation (RPVI). Segmental APWs were obtained with Unetixs Multilab 2-LHS and 2-CP physiologic testing equipment (Unetixs Vascular, Inc., North Kingstown, R.I.).

All lower extremity arterial physiologic evaluations were performed on patients in a supine position following ten minutes of rest. Thigh APWs were obtained at the superior most aspect of the thigh, calf waveforms were acquired inferior to the knee, and all waveforms were obtained with a bladder cuff size of 11.4×40.6 cm. Standard lower extremity segmental pressure protocol and ankle-brachial indices were performed in all examinations bilaterally; however, the data analysis was limited to APWs.

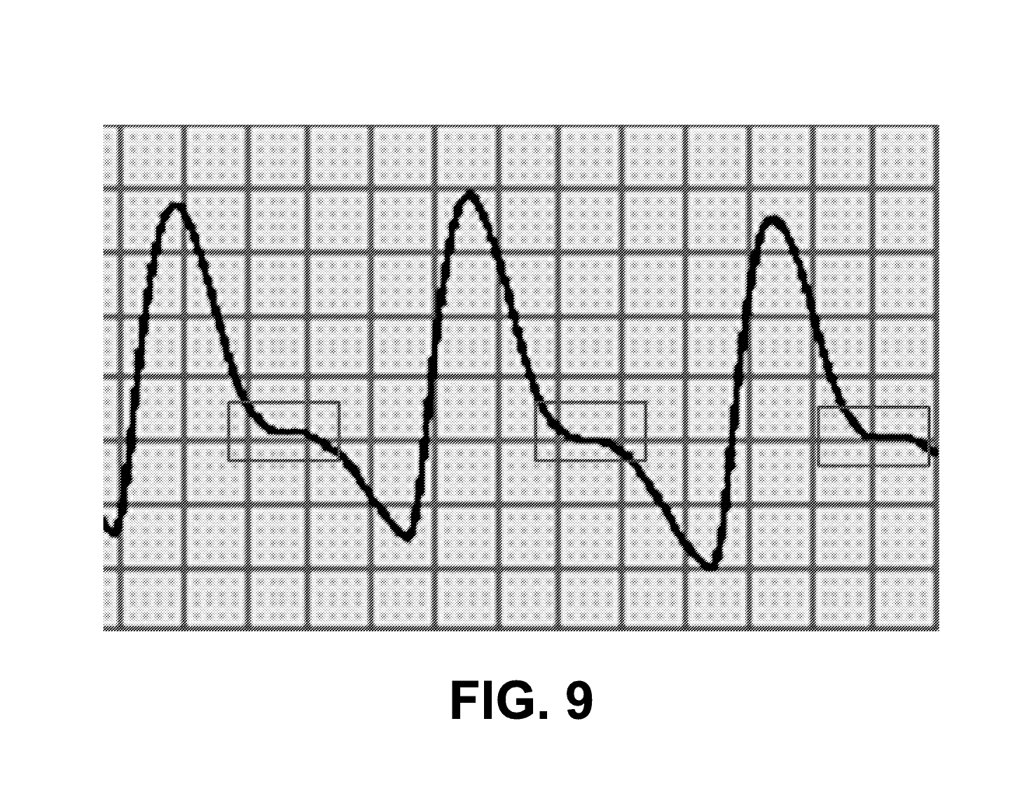

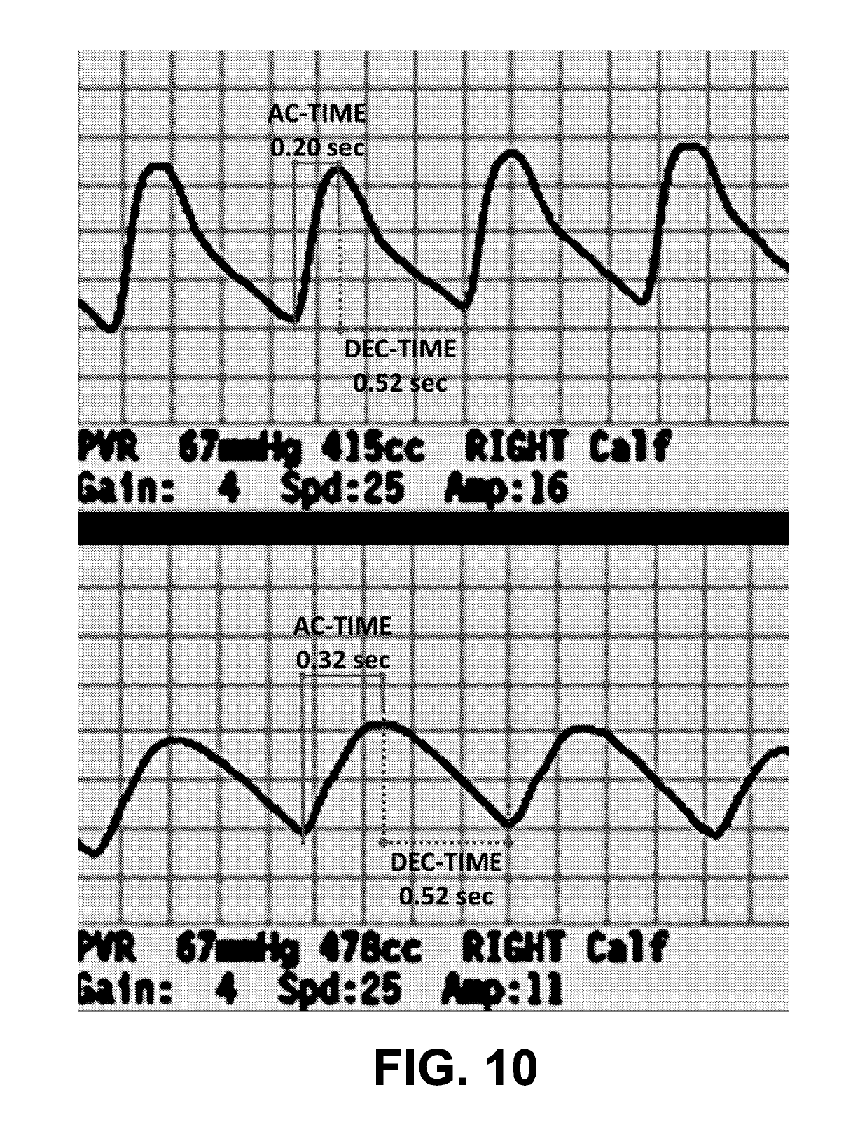

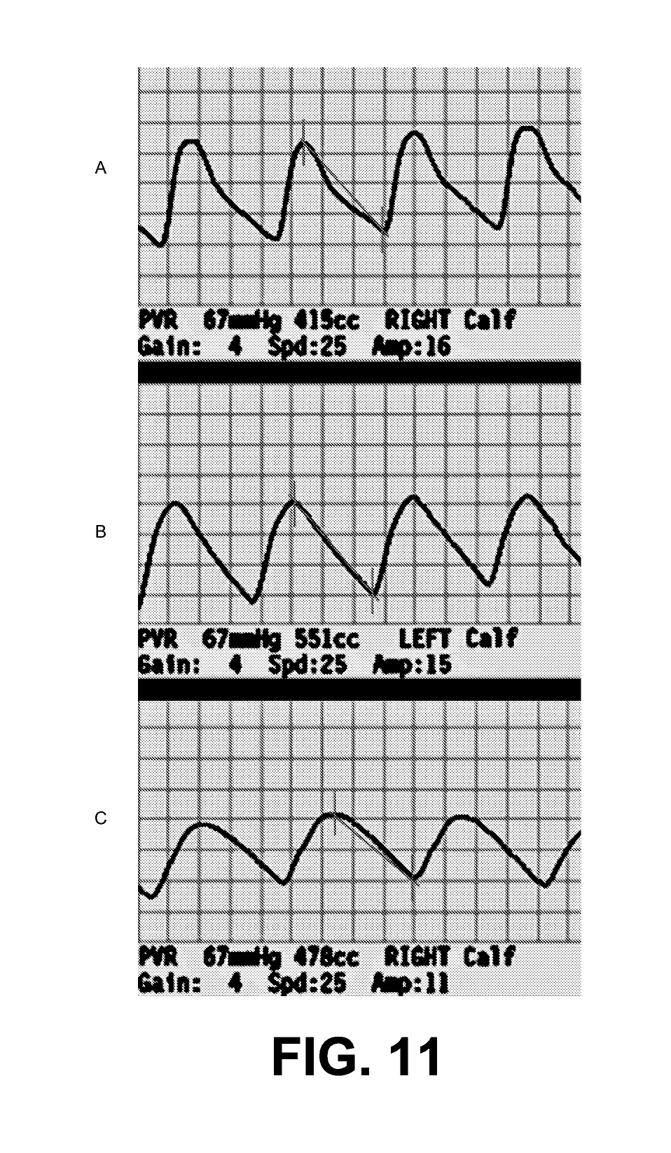

Unetixs calibrated pulse volume recording was utilized to standardize cuff pressure and air volume; chart recorder speed was 25 mm/sec for all measurements. Thigh and calf waveforms that were most representative of the waveform cycle were analyzed for the following characteristics:
1. Dicrotic notch (NOTCH)—presence or absence of a pronounced dicrotic notch (FIG. 9).
2. Acceleration time (AC-TIME)—in seconds, from onset of systole to mid-peak systole rounded to the nearest 0.04 seconds (solid line of FIG. 10).
3. Deceleration time (DEC TIME)—in seconds, from the mid-peak systole to the end of diastole rounded to the nearest 0.04 seconds (dotted line of FIG. 10).
4. Downslope curvature (CURV)—the direction of the waveform downslope curvature in relation to the reference line drawn from mid-peak systole to end diastole.
   a. Inward CU RV—≧50% wave curvature toward the baseline (FIG. 11A).
   b. Even CURV—<50% wave curvature toward or away from baseline (FIG. 11B)
   c. Outward CURV—50% wave curvature away from baseline (FIG. 11C).

Two physicians were responsible for interpretation of the angiograms and were blinded to the APW data. Angiogram comparison to thigh and calf APW data were based on disease location. Thigh waveforms were evaluated when there was ≧50% diameter obstruction at the common femoral artery level or above and/or when there was <50% stenosis of the superficial femoral and/or popliteal artery. Calf waveforms were analyzed when there was ≧50% diameter obstruction of the superficial femoral and/or popliteal artery and <50% stenosis of all vessels above the level of the common femoral artery. Tibioperoneal disease (RUNOFF) disease did not exclude thigh or calf APW analysis in extremities with the previously defined INFLOW and OUTFLOW disease parameters.

NOTCH and CU RV were calculated as percentages; mean and standard deviation for AC-TIME and DEC-TIME were averaged and statistical comparison at the thigh and calf levels was performed with QuickCalcs GraphPad software. A value of P<0.05 was used as the level of statistical significance.

### Results

APW and angiogram data were available for 36 patients (43 limbs); 44% (16/36) were females and 56% (20/36) were males, and the average patient age was 66 years (range 43 to 91). The table below summarizes the APW characteristics for thigh and calf waveforms in extremities with significant INFLOW and OUTFLOW disease.

| Air Plethysmography | Thigh | Calf |
|---|---|---|
| Waveforms | (n = 22) | (n = 21) |
| Dicrotic Notch | | |
| Present | 0 | 0 |
| Absent | 21 | 20 |
| Equivocal | 1 | 1 |
| Downslope (CURV) | | |
| Inward | 7 (32%) | 6 (29%) |
| Even | 12 (55%) | 11 (52%) |
| Outward | 3 (14%) | 4 (19%) |
| Acceleration Time | 0.29 ± 0.57 | 0.26 ± 0.052 |
| (AC-TIME) | | |
| Deceleration Time | 0.55 ± 0.127 | 0.55 ± 0.113 |
| (DEC-TIME) | | |

Seventeen patients (22 extremities) had thigh APW data equally distributed to the right (11/22) and left (11/22) extremity. Mean INFLOW obstruction was 85% diameter reduction (range 65%-100%); 36% (8/22) had common iliac, 18% (4/22) external iliac, and 9% (2/22) had common femoral artery obstruction singularly or in combination. Additionally, 82% (18/22) had tibioperoneal obstructions in one or more runoff vessels.

There were 19 patients (21 extremities) with calf APW data: 38% (8/21) were right and 62% (13/21) left extremities. Mean OUTFLOW obstruction was 92%; 71% (15/21) had superficial femoral, 14% (3/21) had popliteal, and 14% (3/21) had combined superficial femoral and popliteal artery obstruction. Additionally, 86% (18/21) had tibioperoneal obstruction in one or more runoff vessels.

Mean AC-TIME and DEC-TIME for thigh waveforms were 0.29+0.057 and 0.56±0.127 seconds, respectively. Mean AC-TIME and DEC-TIME for calf waveforms were 0.26±SD and 0.55±SD seconds, respectively. There were statistically significant differences between AC-TIME and DEC-TIME for both groups (P<0.0001). Thigh waveform DEC-TIME averaged 2.0 times longer than the AC-TIME (range 1.3 to 3.8); calf waveform DEC-TIME averaged 2.1 times longer than the AC-TIME (range 1.3 to 3.2).

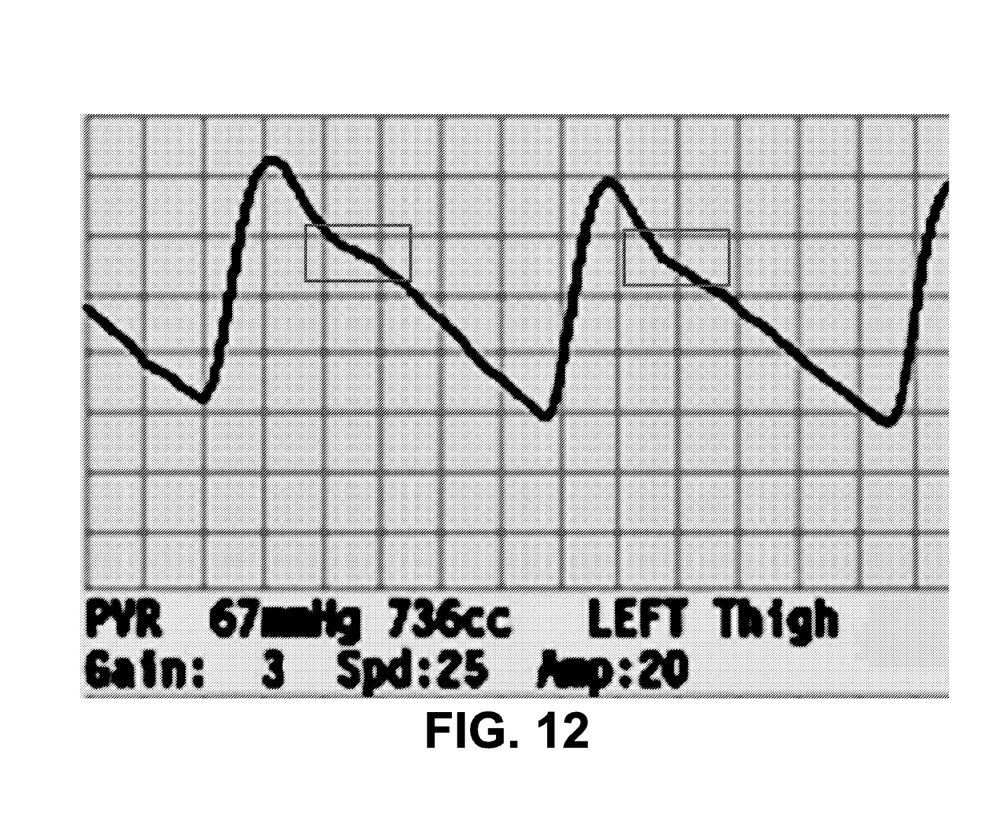

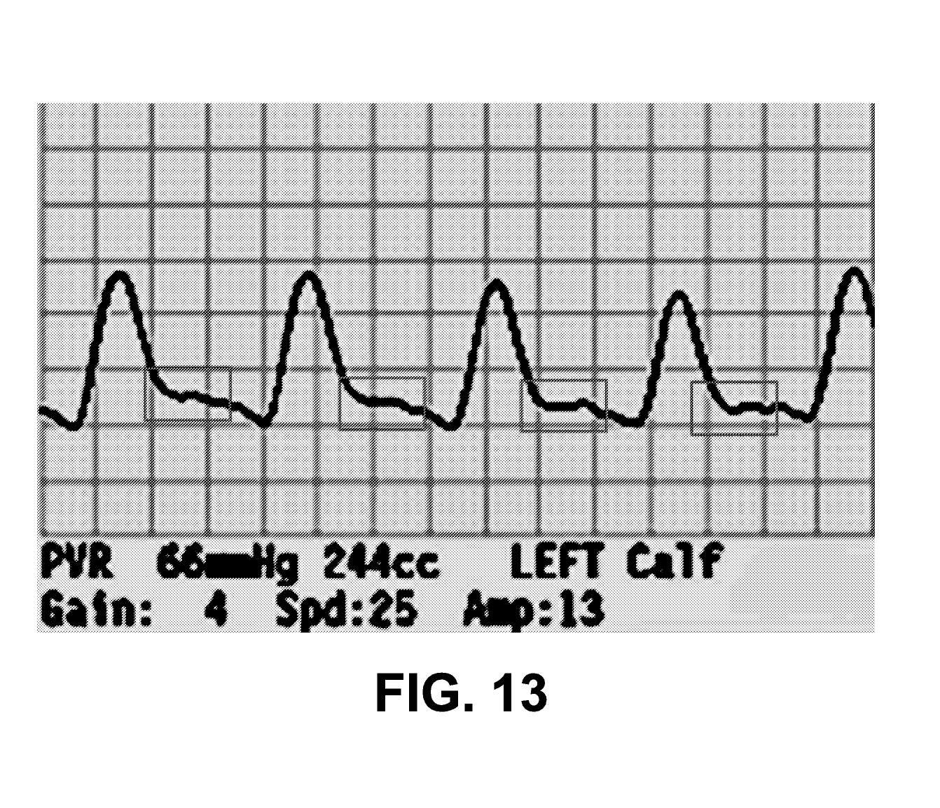

The majority of the thigh and calf waveforms had either an inward or even CURV. Thigh waveform CURV was 32% (7/22) inward, 54% (12/22) even, and 14% (3/22) outward; calf waveform CURV was 29% (6/21) inward, 52% (11/21) even, and 19% (4/21) outward. While no conspicuous dicrotic notch was noted in any APW, one thigh (FIG. 12) and one calf (FIG. 13) APW had equivocal evidence for this characteristic.

### Example 3

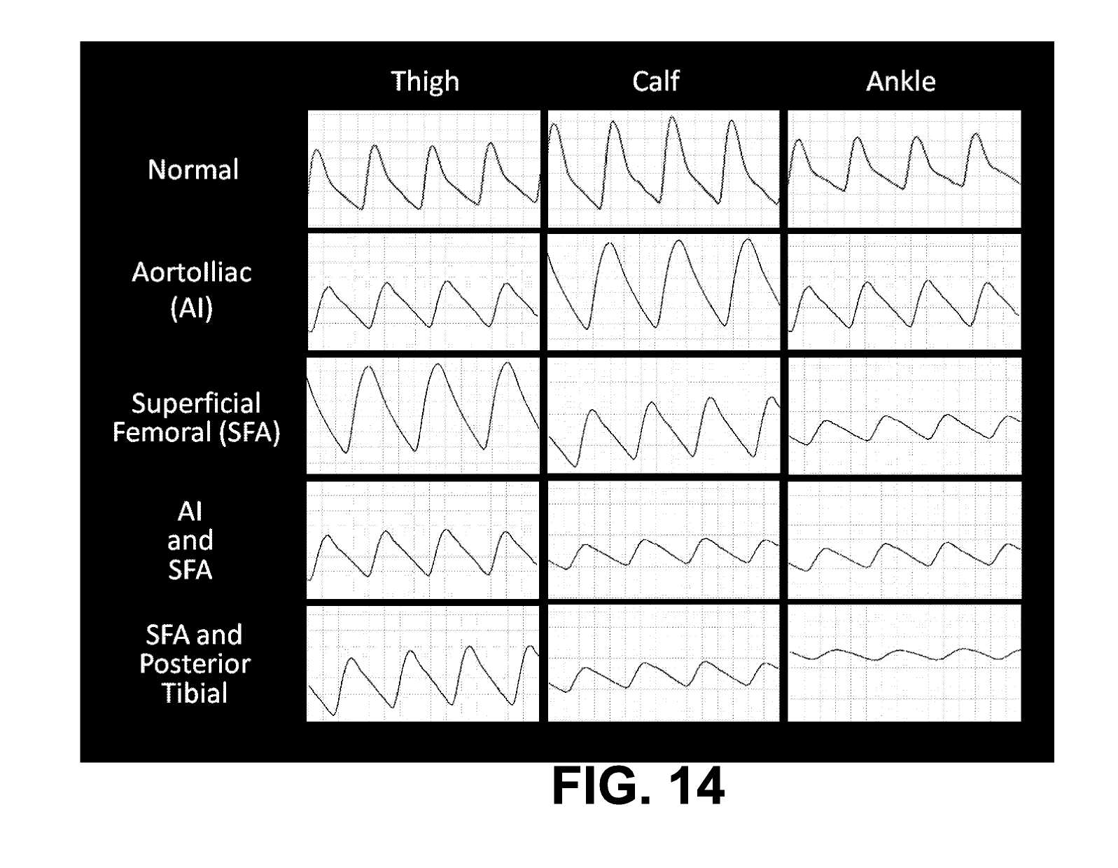

This example illustrates a comparison between subjective (pattern recognition, as shown in FIG. 14) and objective APW analysis methods.

### Methods

Vascular laboratory patients with arterial segmental waveform/pressure analyses and arteriography within thirty days of their noninvasive procedure were investigated. Patients with prior aortoiliac intervention were excluded from the analysis. Institutional review board approval for a retrospective review was obtained through our organization.

Systolic limb pressures and air plethysmographic waveforms were obtained with Unetixs Multilab 2-LHS and 2-CP physiologic testing equipment and 8 or 5 MHz bi-directional, CW Doppler transducers (Unetixs Vascular, Inc., North Kingstown, R.I.). Segmental upper arm brachial and lower extremity thigh, below knee, posterior tibial and dorsalis pedis ankle pressures were performed with patients in the supine position following ten minutes of rest. Thigh pressures were obtained at the uppermost segment of the thigh, using a cuff bladder size of 11.4×40.6 cm. Upper thigh and below knee (calf) pressure measurements were obtained from the ankle level posterior tibial (PT) and dorsalis pedis (DP) artery with the highest systolic pressure. Thigh, calf and ankle pressure indices referenced the higher of the two brachial pressure measurements.

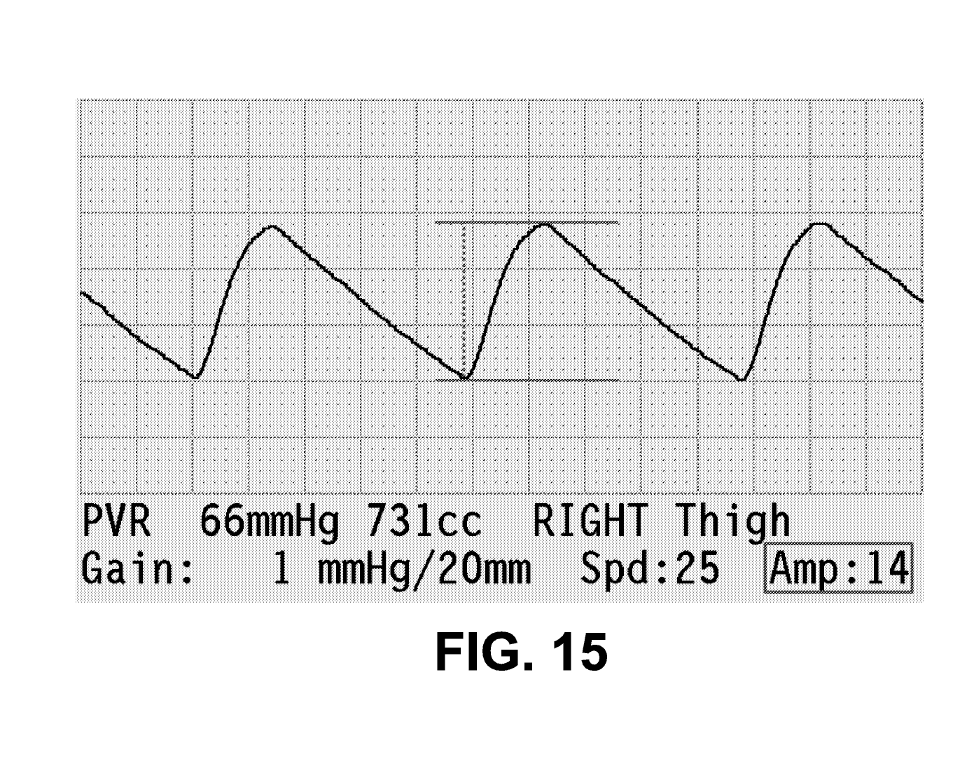

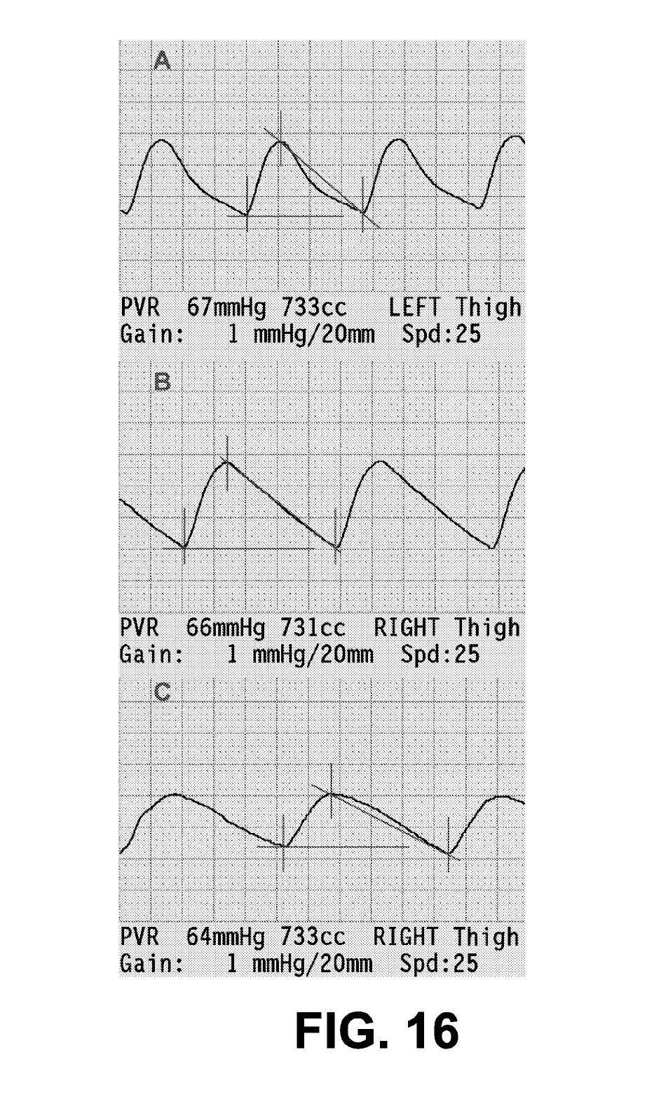

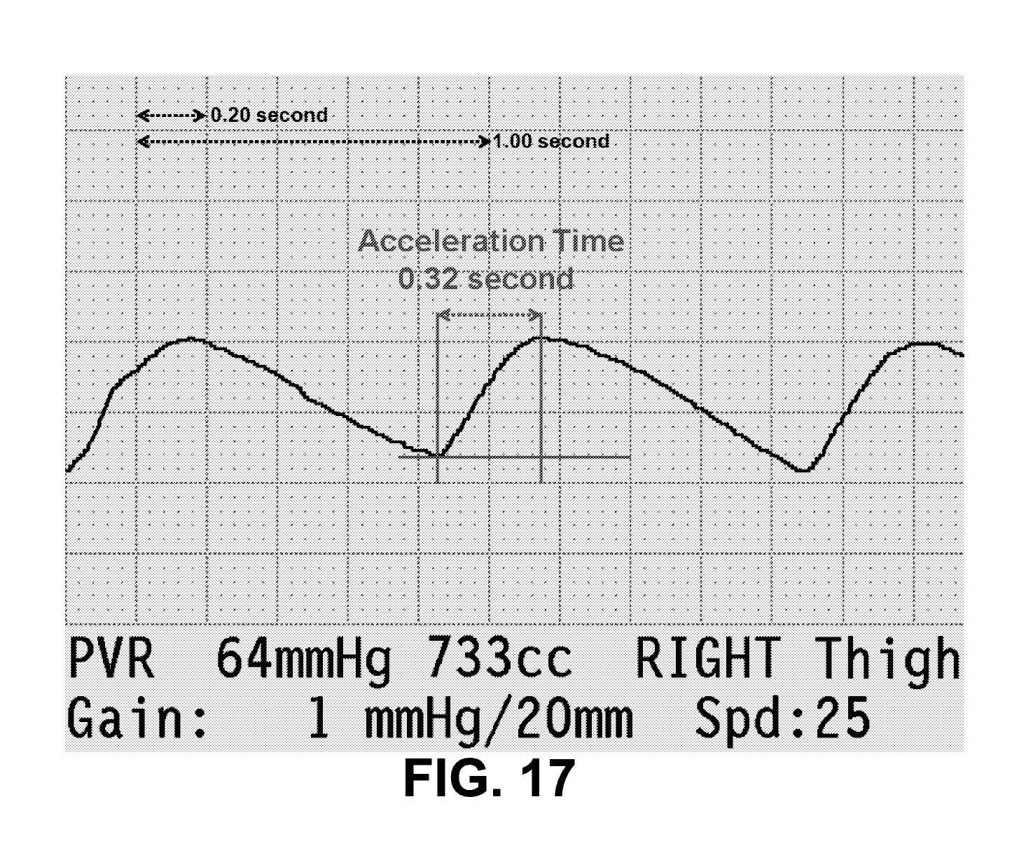

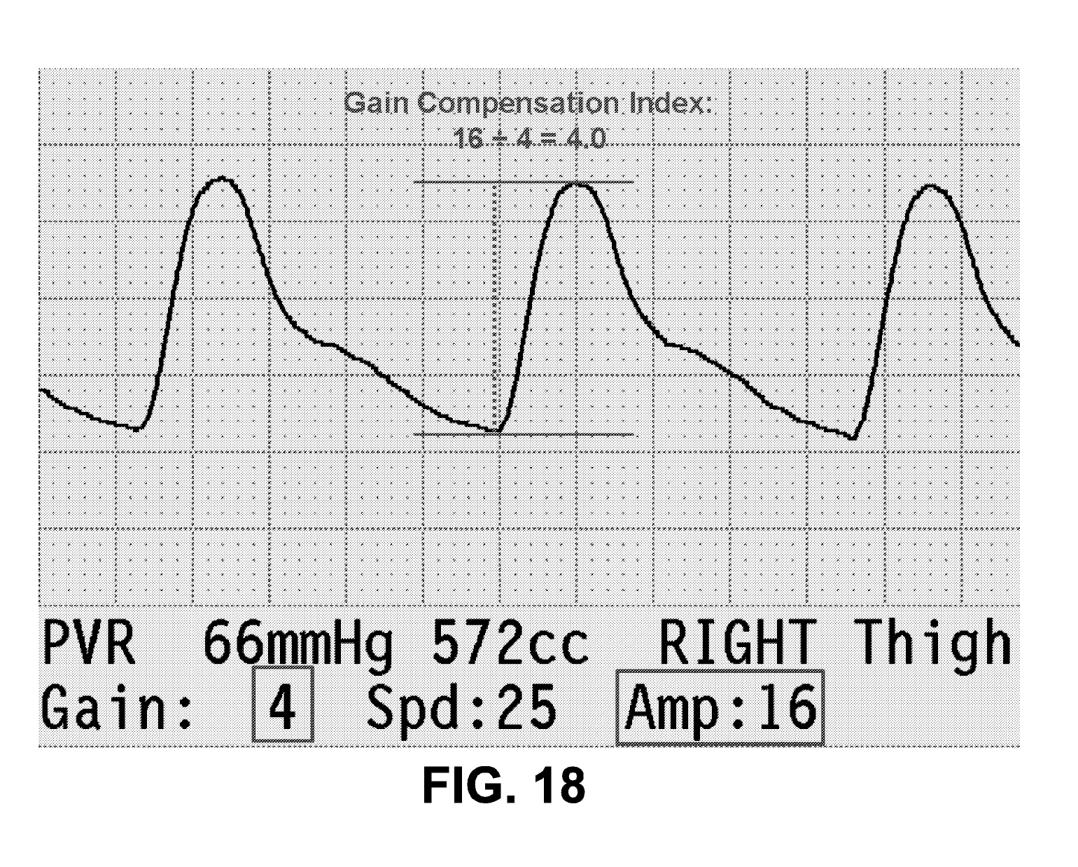

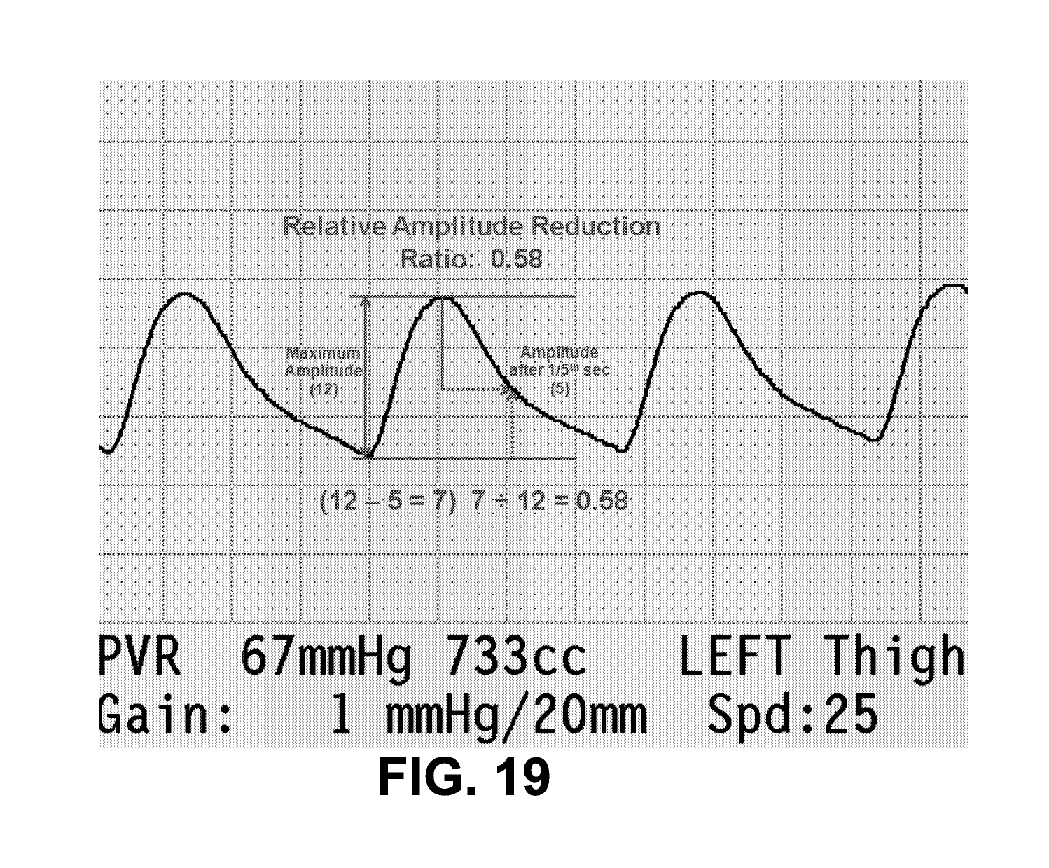

Calibrated pulse volume recording was employed to standardize cuff pressure and air volume. Relative waveform gain varied but was standardized for each patient at the thigh, calf and ankle levels; chart recorder speed was 25 mm/sec for all measurements. The upper thigh waveform most representative of the displayed waveform cycle was evaluated for the following characteristics:
1. Maximum systolic amplitude (AMP) deflection in millimeters (FIG. 15).
2. Down-slope curvature (CURV) based on the direction of the dicrotic limb curvature in relationship to a reference line drawn from mid-peak systole to end diastole.
   a. Inward CURV—inward deflection of 50% or greater from baseline (FIG. 16A).
   b. Even CURV—inward and/or outward deflection of less than 50% (FIG. 16B).
   c. Outward CURV—an outward deflection of 50% or greater from baseline (FIG. 16C).
3. Acceleration time (TIME), in seconds, from the onset of systole to mid-peak systole and rounded to the nearest 0.04 second (FIG. 17).
4. Gain compensation index (GAIN) expressed as a ratio by dividing maximum systolic waveform amplitude by relative gain (FIG. 18).
5. Relative amplitude reduction (RAR) expressed as a ratio: maximum peak systolic waveform amplitude from the onset of systole minus the downslope waveform amplitude one-fifth of a second after mid-peak systole, divided by maximum peak systolic waveform amplitude and rounded to the nearest 0.04 second (FIG. 19).

Two physicians shared responsibility for interpreting the arteriograms and were blinded to the noninvasive data. Each limb was classified as negative or positive for hemodynamically significant INFLOW disease. Positive INFLOW was defined as greater than 50 percent diameter stenosis or occlusion in one or more of the following vessels: abdominal aorta, common iliac, external iliac or common femoral artery. Evaluation for INFLOW based on the APW pattern recognition method was performed by an American Registry for Diagnostic Medical Sonography (ARDMS) Registered Physician in Vascular Interpretation (RPVI) with more than thirty years experience in noninvasive arterial physiologic testing and blinded to the arteriography, segmental pressure—thigh, calf, PT, DP, and ABI data.

Statistical analysis was performed using SAS 9.2 (TS level 2M3, XP_PRO platform, SAS Institute, Cary, N.C.). INFLOW analyses included: objective APW waveform versus pattern recognition, and thigh, ankle, PT, DP pressure and ABI data. For all analyses, a P value of less than 0.05 was used to define statistical significance.

### Results

Vascular laboratory and angiography data were available from 78 patients; eight patients were excluded due to prior aortoiliac intervention. 70 patients were investigated; 64% were males (45/70) and 36% females (25/70). Average patient age was 71 years old, ranging from 35 to 95 years. Diabetics represented 57% (40/70) and non-diabetics 43% (30/70) of the patient cohort. There were 37 bilateral and 33 unilateral extremity evaluations with appropriate clinical indications of claudication (33%), limb ischemia (24%), rest pain (17%), ulceration (17%), gangrene (7%) and suspected abdominal aortic aneurysm (1%).

### Angiography

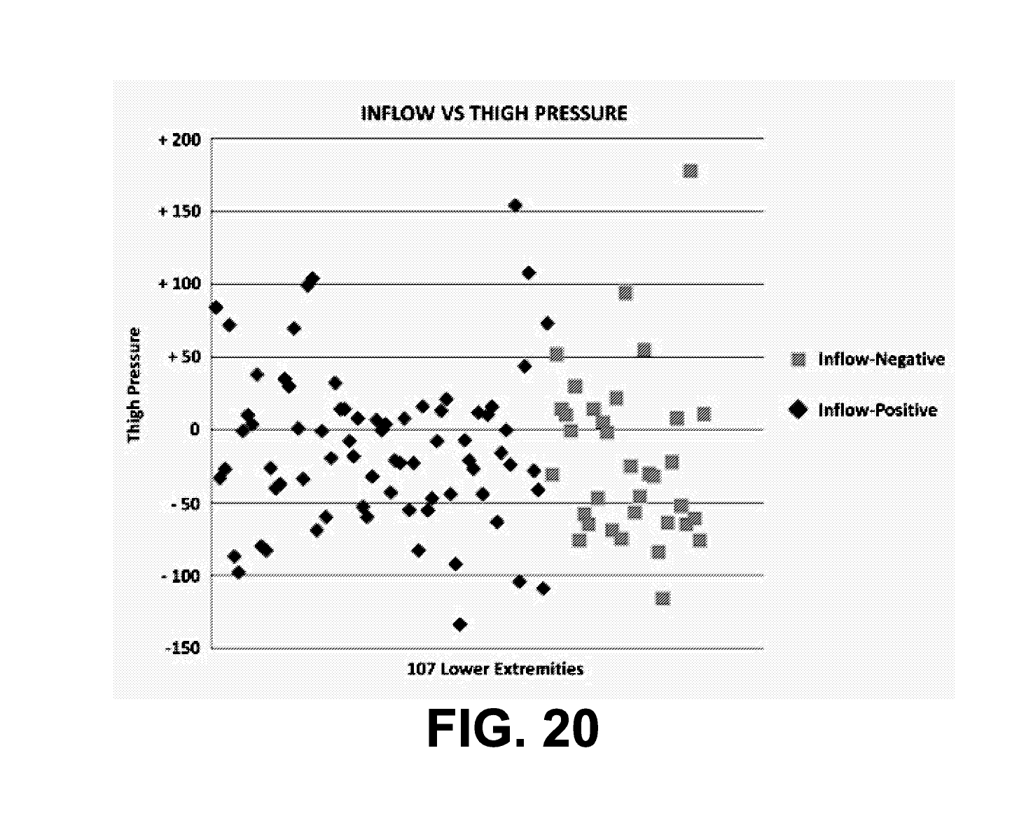

107 extremities were analyzed; 50% (53/107) right and 50% (54/107) left. Thigh cuff pressure measurements averaged 14 mmHg less than the highest brachial systolic cuff pressure and are summarized in FIG. 20. Average of the higher of the PT or DP ABI was 0.82; the combined average of both the PT and DP ABI was 0.70.

73 extremities (68%) were negative for INFLOW: four <50% extremities, 15 with single level femoropopliteal disease, nine with outflow (tibioperoneal) disease and 45 extremities with concomitant femoropopliteal and tibioperoneal disease. Thigh cuff pressure measurements averaged 12 mmHg less than the highest brachial systolic cuff pressure. Average of the higher of the PT or DP ABI was 0.84; the combined average of both the PT and DP ABI was 0.72. A discernible dicrotic notch was present in 23% (17/73) waveforms. Downslope curvature toward the baseline was evident in 73% (53/73), even curvature 25% (18/73) and outward curvature in 3% (2/73) of limbs negative for INFLOW.

34 extremities (32%) were positive for INFLOW: seven single level inflow, six multilevel with associated femoropopliteal disease, seven multilevel with associated outflow disease, and 14 multilevel with concomitant femoropopliteal and outflow disease. Thigh cuff pressure measurements averaged 19 mmHg less than the highest brachial systolic cuff pressure. Average of the higher of the PT or DP ABI was 0.78; the combined average of both the PT and DP ABI was 0.67. A dicrotic notch was not present in any extremity waveform positive for INFLOW. Downslope curvature toward the baseline was evident in 29% (10/34), even curvature 41% (14/34) and outward curvature in 29% (10/34) of limbs positive for INFLOW.

There were 43% (46/107) non-diabetic extremities, with 30 negative and 16 (35%) positive for INFLOW, and 57% (61/107) diabetic extremities, with 43 negative and 18 (30%) positive for INFLOW. The level(s) of disease, thigh pressures and ABI data are summarized in the Table below.

| | Non-Diabetic | Diabetic |
|---|---|---|
| | (n = 46) | (n = 61) |
| Inflow Negative | 30 | 43 |
| Normal (<50%) | 1 | 3 |
| Femoropopliteal | 8 | 7 |
| Outflow | 5 | 4 |
| Multi-Level: | 16 | 29 |
| Femoropoplital + Outflow | | |
| Brachial-Thigh Cuff Pressure | −14 | −11 |
| Difference (mmHg) | | |
| Highest (PT or DP) ABI | 0.76 | 0.90 |
| Average | | |
| Combined (PT + DB) ABI | 0.65 | 0.77 |
| Average | | |
| Inflow Positive | 16 | 18 |
| Inflow (CFA or above) | 4 | 3 |
| Multi-Level: | 3 | 3 |
| Inflow + Femoropopliteal | | |
| Multi-Level: Inflow + Outflow | 4 | 3 |
| Multi-Level: | 5 | 5 |
| Inflow + Femoropoplital + Outflow | | |
| Brachial-Thigh Cuff Pressure | −24 | −15 |
| Difference (mmHg) | | |
| Highest (PT or DB) ABI | 0.67 | 0.88 |
| Average | | |
| Combined (PT + DB) ABI | 0.63 | 0.70 |
| Average | | |

No statistically significant differences were noted between groups for total extremities tested (P=0.711), inflow negative or positive (P=0.561) and level of disease, (P=0.816).

### Thigh APW Analysis

There were significant differences in the objective versus subjective APW interpretation method, P=0.0110. An algorithm for the classification of INFLOW based on objective thigh waveform characteristics is defined in the Table below.

| | INFLOW NEGATIVE |
|---|---|
| | Non-Diabetic |
| CURV-IN | TIME ≠ 0.28 seconds or RAR > 0.50 |
| CURV-EVEN | TIME < 0.28 seconds |
| CURV-EVEN | TIME = 0.28 seconds and GAIN > 3.0 |
| | INFLOW POSITIVE |
| | Non-Diabetic |
| CURV-IN | TIME = 0.28 seconds and RAR < 0.50 |
| CURV-EVEN | TIME > 0.28 seconds |
| CURV-EVEN | TIME = 0.28 seconds and GAIN < 3.0 |
| CURV-OUT | Any CURV-OUT = Positive Inflow |
| | INFLOW NEGATIVE |
| | Diabetic |
| CURV-IN | GAIN NOT ≦ 2.5 with RAR < 0.30 and GAIN |
| | NOT < 5 with RAR = 0.50 |
| CURV-EVEN | GAIN ≦ 6 and AMP ≧ 7 |
| | INFLOW NEGATIVE |
| | Non-Diabetic |
| CURV-IN | GAIN ≦ 2.5 and RAR < 0.30 |
| CURV-IN | GAIN < 5 and RAR = 0.50 |
| CURV-EVEN | GAIN ≧ 7 or AMP ≦ 6 |
| CURV-OUT | Any CURV-OUT = Positive Inflow |

A comparison of sensitivity, specificity, positive and negative predictive value (PPV/NPV) and accuracy percentages based on objective waveform characteristics and subjective pattern recognition are summarized in the Table below.

| HEMODYNAMICALLY | | |
|---|---|---|
| SIGNIFICANT (>50% | | |
| Diameter) INFLOW | Objective APW | Subjective APW |
| DISEASE | Analysis | Analysis |
| Sensitivity | 85% | 53% |
| Specificity | 93% | 85% |
| Positive Predictive Value | 85% | 62% |
| Negative Predictive Value | 93% | 80% |
| Accuracy | 91% | 75% |

Objective waveform parameters associated with pattern recognition interpretations are presented for comparison in the Table below.

| | | Objective | Subjective |
|---|---|---|---|
| | | APW | APW |
| Inflow Negative | | | |
| Thigh Cuff APW | | | |
| Relative Gain | | 5.4 | 5.3 |
| Maximum Amplitude (AMP) | | 17.5 | 17.7 |
| Downslope Curvature (CURV) | Inward | 55 | 62 |
| | Even | 18 | 16 |
| | Outward | 0 | 0 |
| Gain Compensation Index (GAIN( | | 3.6 | 3.6 |
| Seconds Rise Time (TIME) | | 0.26 | 0.26 |
| Relative Amplitude Reduction (RAR) | | 0.49 | 0.49 |
| Inflow Positive | | | |
| Thigh Cuff APW | | | |
| Relative Gain | | 5.8 | 6.0 |
| Maximum Amplitude (AMP) | | 10.6 | 8.7 |
| Downslope Curvature (CURV) | Inward | 8 | 1 |
| | Even | 14 | 16 |
| | Outward | 12 | 12 |

| | | |
|---|---|---|
| Gain Compensation Index (GAIN( | 2.0 | 1.6 |
| Seconds Rise Time (TIME) | 0.32 | 0.32 |
| Relative Amplitude Reduction (RAR) | 0.34 | 0.34 |

### Thigh Pressure Analysis

Objective APW analysis was significantly better for defining INFLOW when compared to brachial to thigh pressure differences of >20 mmHg and >0 mmHg differences, P=0.000 respectively. Sensitivity, specificity, PPV, NPV and accuracy percentages of brachial-thigh pressure differences and APW analysis are summarized in the Table below.

| HEMODYNAMICALLY | | | |
|---|---|---|---|
| SIGNIFICANT (>50% | | Brachial | Brachial |
| Diameter) INFLOW | Objective | to Thigh | to Thigh |
| DISEASE | APW | Pressure (20+) | Pressure (0+) |
| Sensitivity | 85% | 59% | 65% |
| Specificity | 93% | 52% | 41% |
| Positive Predictive | 85% | 36% | 34% |
| Value | | | |
| Negative Predictive | 93% | 73% | 71% |
| Value | | | |
| Accuracy | 91% | 54% | 49% |

From the above description, those skilled in the art will perceive improvements, changes and modifications. Such improvements, changes and modifications are within the skill of one in the art and are intended to be covered by the appended claims.

What is claimed is:

1. A system, comprising:
a memory to store computer-executable instructions; and
a processor to execute the computer-executable instructions to at least:
receive an air plethysmographic waveform (APW) taken from a subject;
determine a characteristic of the APW based on an analysis of the APW; and
provide a suggested diagnosis for the subject based on the characteristic of the APW.

2. The system of claim 1, wherein the processor executes the computer-executable instructions to compare the characteristic of the APW to a control characteristic of a control APW, and
wherein the suggested diagnosis is provided based on the comparison.

3. The system of claim 2, wherein the APW is taken from a location on the subject's body, and
wherein the control APW corresponds to an APW taken at another location on the subject's body.

4. The system of claim 3, wherein the APW and the control APW are taken from different segments of the subject's body, and
wherein the comparison is a multi-segment comparison.

5. The system of claim 2, wherein the control APW corresponds to a stored historical APW with the control characteristic known.

6. The system of claim 1, wherein the suggested diagnosis is provided on a display device.

7. The system of claim 1, wherein the characteristic of the APW comprises at least one of: a presence of a dicrotic notch, a relative amplitude, an acceleration time, a deceleration time, and a downslope curvature.

8. A method, comprising:
receiving, by a system comprising a processor, an air plethysmographic waveform (APW) taken from a subject;
determining, by the system, a characteristic of the APW based on an analysis of the APW; and
providing, by the system, a suggested diagnosis for the subject based on the characteristic of the APW.

9. The method of claim 8, wherein the suggested diagnosis is one of normal, mild disease, moderate disease, and severe disease.

10. The method of claim 8, wherein the suggested diagnosis is related to a peripheral artery disease (PAD).

11. The method of claim 8, wherein the suggested diagnosis comprises a probability of a vascular obstruction.

12. The method of claim 8, further comprising comparing, by the system, the characteristic of the APW to a control characteristic of a control APW,
wherein the suggested diagnosis is provided based on the comparison.

13. The method of claim 12, wherein the control characteristic corresponds to a normal diagnosis.

14. The method of claim 12, wherein the APW and the control APW correspond to different segments of the patient's body.

15. The method of claim 14, wherein the APW and the control APW are received together in a combined signal, and
wherein the receiving the APW further comprises splitting the APW from the control APW.

16. The method of claim 8, wherein the characteristic of the APW comprises at least one of: a presence of a dicrotic notch, a relative amplitude, an acceleration time, a deceleration time, and a downslope curvature.

17. A device, comprising:
an air plethysmograph recording device coupled to a computing device,
wherein the computing device comprises:
a memory to store computer-executable instructions; and
a processor to execute the computer-executable instructions to at least:
receive an air plethysmographic waveform (APW) taken from a subject from the air plethysmograph recording device;
determine a characteristic of the APW based on an analysis of the APW; and
provide a suggested diagnosis for the subject based on the characteristic of the APW.

18. The device of claim 17, further comprising a display device to display a report comprising the suggested diagnosis for the subject.

19. The device of claim 18, wherein the report comprises a probability of an obstruction in an artery reflected in the APW.

20. The device of claim 17, wherein the characteristic of the APW comprises at least one of: a presence of a dicrotic notch, a relative amplitude, an acceleration time, a deceleration time, and a downslope curvature.
